(19) United States
(12) Patent Application Publication
(10) Pub. No.: US 2023/0240798 A1
(43) Pub. Date: Aug. 3, 2023

(54) DENTAL COMPONENTS

(71) Applicant: THE TEXAS A&M UNIVERSITY SYSTEM, College Station, TX (US)

(72) Inventors: Amirali ZANDINEJAD, College Station, TX (US); Marta Revilla LEON, College Station, TX (US)

(21) Appl. No.: 18/001,604

(22) PCT Filed: Jun. 18, 2021
(86) PCT No.: PCT/US2021/038035

§ 371 Date: Dec. 13, 2022

Related U.S. Application Data
(60) Provisional application No. 63/040,578, filed on Jun. 18, 2020.

(51) Int. Cl. A61C 5/77

(52) U.S. Cl. CPC A61C 5/77

(57) ABSTRACT

A multilayer crown includes an outer layer and an inner layer. The outer layer may be formed of a first polymeric material. The inner layer may be formed of a second polymeric material that is different from the first polymeric material. The inner layer may be arranged to contact a tooth so that the inner layer is located between the outer layer and the tooth.

### CROSS-REFERENCE TO RELATED APPLICATIONS

This application claims priority under 35 U.S.C. § 119(e) to U.S. Provisional Application Nos. 63/040,578 filed on Jun. 18, 2020, the entire disclosure of which is incorporated herein by reference.

### BACKGROUND

Additively manufacturing (AM) technologies provide a new manufacturing method for processing polymers, metal, and ceramics which facilitate the fabrication of many dental devices including dental restorations. AM methods provide the capability of manufacturing complex geometries and designs that cannot be produced with subtractive methods. Furthermore, AM technologies have been investigated for processing different materials at the same time, which have the potential to introduce multifunctionality on AM devices.

Among the different AM technologies, material jetting, also called polyjet printing, is a process in which droplets of feedstock material are selectively deposited and polymerized by an UV light, layer-by-layer, until the 3D device is built. Material jetting printers deposit the layers of the dental photopolymer resin and typically the support material to partially surround it which enables the manufacturing of different materials with different color and/or properties. Material jetting 3D printers require minimum post-processing procedures where the supportive material is removed by heating or water washout.

A bio-inspired restoration is an approach in biomedical engineering that aims to artificially manufacture a dental restoration which reproduces the structure and properties of dental tissues. AM technologies and restorative materials are still in continuous development, aspiring to be able to manufacture a bio-inspired dental restoration; however, material jetting technologies may be able to manufacture a multi-layer dental restoration by combining polymers with different colors, viscosities, and mechanical properties.

### SUMMARY

According to one aspect of the present disclosure, a multilayer dental restoration comprises an outer layer and an inner layer. In some embodiments, the outer layer formed of a first polymeric material. In some embodiments, the inner layer formed of a second polymeric material that is different from the first polymeric material. In some embodiments, the inner layer is arranged to contact a tooth so that the inner layer is located between the outer layer and the tooth. In some embodiments, the inner layer has a hardness that is lower than a hardness for the outer layer. In some embodiments, the inner layer has an elasticity that is higher than an elasticity of the outer layer so that the multilayer dental restoration can compensate up to about 40%, up to about 30%, up to about 25%, or up to about 15% of manufacturing error of the dental crown.

According to another aspect of the present disclosure, a method for forming a multilayer dental restoration, comprises providing a first material to form a first layer; providing a second material that has different properties than the first material to form a second layer; curing the first material to form a first cured material and curing the second material to form a second cured material. In some embodiments, the first material is arranged to form an outer layer of the multilayer dental crown. In some embodiments, the second material is arranged to form an inner layer of the multilayer dental restoration.

According to another aspect of the present disclosure, a dental component comprises a porous layer and a non-porous layer. In some embodiments, the porous layer forms an inner layer of the dental component. In some embodiments, the non-porous layer forms an outer layer of the dental component.

According to another aspect of the present disclosure, a dental implant comprises an abutment, a crown, and a post. In some embodiments, the abutment comprises a multilayer material and is configured to receive a dental crown. In some embodiments, the post is located spaced apart from the abutment. In some embodiments, the post couples the abutment to a jaw bone. In some embodiments, the multilayer material of the abutment comprises a porous material configured to contact soft tissue of a patient.

According to another aspect of the present disclosure, a post for a tooth root comprises a shaft. In some embodiments, the shaft is formed of a lattice material. In some embodiments, the shaft is configured to extend through a prepared tooth to couple a dental crown to the prepared tooth. In some embodiments, the post is formed of a metal, a ceramic, a glass ceramic, or a polymeric material.

Additional embodiments, features, and advantages of the disclosure will be apparent from the following detailed description and through practice of the disclosure. The process and compounds of the present disclosure can be described as embodiments in any of the following enumerated clauses. It will be understood that any of the embodiments described herein can be used in connection with any other embodiments described herein to the extent that the embodiments do not contradict one another.

1. A multilayer dental restoration, comprising

an outer layer formed of a first polymeric material,

an inner layer formed of a second polymeric material that is different from the first polymeric material, the inner layer arranged to contact a tooth so that the inner layer is located between the outer layer and the tooth,

wherein the inner layer has a hardness that is lower than a hardness for the outer layer and the inner layer has an elasticity that is higher than an elasticity of the outer layer so that the multilayer dental restoration can compensate up to about 25% of manufacturing error of the multilayer dental restoration.

2. The multilayer dental restoration of any one of the preceding clauses, wherein the inner layer has a flexural modulus of about 1500 to 2500 MPa.

3. The multilayer dental restoration of any one of the preceding clauses, wherein the outer layer has a flexural modulus of about 2500 MPa to about 6000 MPa.

4. The multilayer dental restoration of any one of the preceding clauses, wherein the inner layer has a flexural strength of about 35 to 50 MPa.

5. The multilayer dental restoration of any one of the preceding clauses, wherein the outer layer has a flexural strength of about 65 MPa to about 150 MPa.

6. The multilayer dental restoration of any one of the preceding clauses, wherein the inner layer has a modulus of elasticity of about 1,500 MPa to 2,500 MPa.

7. The multilayer dental restoration of any one of the preceding clauses, wherein the outer layer has a modulus of elasticity of about 2,500 MPa to about 6,000 MPa.

8. The multilayer dental restoration of any one of the preceding clauses, wherein the inner layer has an elongation at break of more than 20%.

9. The multilayer dental restoration of any one of the preceding clauses, wherein the outer layer has an elongation of break of about 5% to about 20%.

10. The multilayer dental restoration of any one of the preceding clauses, wherein the inner layer is about 10% to about 75% by volume of the multilayer dental restoration.

11. The multilayer dental restoration of any one of the preceding clauses, wherein the first polymeric material is a composite.

12. The multilayer dental restoration of any one of the preceding clauses, wherein the first polymeric material comprises triethylene glycol dimethacrylate (TEGDMA), Bis-GMA, Urethane dimethacrylate, or poly methylmethacrylate.

13. The multilayer dental restoration of any one of the preceding clauses, wherein the first polymeric material comprises a filler.

14. The multilayer dental restoration of clause 13, wherein the filler comprises a glass filler, a ceramic, or a combination thereof.

15. The multilayer dental restoration of clause 14, wherein the ceramic comprises zirconia, alumina, or a combination thereof.

16. The multilayer dental restoration of clause 14, wherein the glass filler is such as barium glass.

17. The multilayer dental restoration of any one of the preceding clauses, wherein the outer layer is about 10% to about 70% by volume of the multilayer dental restoration.

18. The multilayer dental restoration of any one of the preceding clauses, wherein the second polymeric material is a composite.

19. The multilayer dental restoration of any one of the preceding clauses, wherein the second polymeric material comprises triethylene glycol dimethacrylate (TEGDMA), Bis-GMA, Urethane dimethacrylate, or poly methylmethacrylate.

20. The multilayer dental restoration of any one of the preceding clauses, wherein the second polymeric material comprises a filler.

21. The multilayer dental restoration of clause 20, wherein the second polymeric material comprises an amount of filler less than an amount of filler in the first polymeric material.

22. The multilayer dental restoration of clause 20, wherein the filler comprises a glass filler, a ceramic, or a combination thereof.

23. The multilayer dental restoration of clause 22, wherein the ceramic comprises zirconia, alumina, or a combination thereof.

24. The multilayer dental restoration of clause 22, wherein the glass filler is such as barium glass.

25. The multilayer dental restoration of any one of the preceding clauses, wherein the second polymeric material is approved for dental use.

26. The multilayer dental restoration of any one of the preceding clauses, wherein the multilayer dental restoration is a crown, an inlay, a veneer, or an onlay.

27. The multilayer dental restoration of any one of the preceding clauses, wherein the multilayer dental restoration is a crown.

28. The multilayer dental restoration of any one of the preceding clauses, wherein the multilayer dental restoration consists of two layers.

29. The multilayer dental restoration of any one of the preceding clauses, wherein the inner layer forms an external surface of the multilayer dental restoration and the outer layer forms the opposite external surface.

30. The multilayer dental restoration of any one of the preceding clauses, wherein the outer layer comprises about 40% to about 80% filler.

31. The multilayer dental restoration of any one of the preceding clauses, wherein the inner layer comprises about 1% to about 35% filler.

32. A method for forming a multilayer dental restoration, comprising:

providing a first material to form a first layer;

providing a second material that has different properties than the first material to form a second layer; and

curing the first material to form a first cured material and curing the second material to form a second cured material,

wherein the first material is arranged to form an outer layer of the multilayer dental restoration and the second material is arranged to form an inner layer of the multilayer dental restoration.

33. The method of clause 32, wherein the inner layer has a hardness that is lower than a hardness for the outer layer.

34. The method of any one of clauses 32-33, wherein the inner layer has an elasticity that is higher than an elasticity of the outer layer.

35. The method of any one of clauses 32-34, wherein the method comprises additively printing a plurality of first layers and a plurality of second layers.

36. The method of any one of clauses 32-35, wherein the step of curing occurs after additively printing each layer of the plurality of layers.

37. The method of any one of clauses 32-36, wherein the step of curing is performed by a light source.

38. The method of any one of clauses 32-37, wherein the step of curing is performed by heat or UV light.

39. The method of any one of clauses 32-38, wherein the first step of providing, the second step of providing, or both is performed by additive printing.

40. The method of any one of clauses 32-39, wherein the steps of additive printing are performed by material jetting, extrusion, lamination, STL, or DLP.

41. The method of any one of clauses 32-40, wherein the steps of additive printing are performed by material jetting.

42. The method of any one of clauses 32-41, wherein the method comprises digitizing a tooth that will be covered by the multilayer dental restoration.

43. The method of any one of clauses 32-42, wherein the inner layer has a flexural modulus of about 1500 to 2500 MPa.

44. The method of any one of clauses 32-43, wherein the outer layer has a flexural modulus of about 2500 MPa to about 6000 MPa.

45. The method of any one of clauses 32-44, wherein the inner layer has a flexural strength of about 35 to 50 MPa.

46. The method of any one of clauses 32-45, wherein the outer layer has a flexural strength of about 65 MPa to about 150 MPa.

47. The method of any one of clauses 32-46, wherein the inner layer has a modulus of elasticity of about 1,500 MPa to 2,500 MPa.

48. The method of any one of clauses 32-47, wherein the outer layer has a modulus of elasticity of about 2,500 MPa to about 6,000 MPa.

49. The method of any one of clauses 32-48, wherein the inner layer has an elongation at break of more than 20%.

50. The method of any one of clauses 32-49, wherein the outer layer has an elongation of break of about 5% to about 20%.

51. The method of any one of clauses 32-50, wherein the inner layer is about 10% to about 75% by volume of the multilayer dental restoration.

52. The method of any one of clauses 32-51, wherein the inner layer is formed of a first polymeric material and the outer layer is formed of a second polymeric material.

53. The method of clause 52, wherein the first polymeric material is a composite.

54. The method of any one of clauses 52-53, wherein the first polymeric material comprises triethylene glycol dimethacrylate (TEGDMA), bis-GMA, urethane dimethacrylate, or poly methylmethacrylate.

55. The method of any one of clauses 52-54, wherein the first polymeric material comprises a filler.

56. The method of any one of clauses 52-55, wherein the filler comprises a glass filler, a ceramic, or a combination thereof.

57. The method of any one of clauses 52-56, wherein the ceramic comprises zirconia, alumina, or a combination thereof.

58. The method of any one of clauses 52-57, wherein the glass filler is such as barium glass.

59. The method of any one of clauses 52-58, wherein the second polymeric material is a composite.

60. The method of any one of clauses 52-59, wherein the second polymeric material comprises triethylene glycol dimethacrylate (TEGDMA), Bis-GMA, urethane dimethacrylate, or poly methylmethacrylate.

61. The method of any one of clauses 52-60, wherein the second polymeric material comprises a filler.

62. The method of any one of clauses 52-61, wherein the filler comprises a glass filler, a ceramic, or a combination thereof.

63. The method of any one of clauses 52-62, wherein the ceramic comprises zirconia, alumina, or a combination thereof.

64. The method of any one of clauses 52-63, wherein the glass filler is barium glass.

65. The method of any one of clauses 52-64, wherein the second polymeric material is approved for dental use.

66. The method of any one of clauses 32-65, wherein the multilayer dental restoration is a crown, an inlay, a veneer, or an onlay.

67. The method of any one of clauses 32-66, wherein the multilayer dental restoration is a crown.

68. The method of any one of clauses 32-67, wherein the multilayer dental restoration consists of two layers.

69. The method of any one of clauses 32-68, wherein the inner layer forms an external surface of the multilayer dental restoration and the outer layer forms the opposite external surface.

70. The method of any one of clauses 32-69, wherein the outer layer is about 10% to about 70% by volume of the multilayer dental restoration.

70a. The method of any one of clauses 32-70, comprising milling the first or second materials or the inner or outer layers.

71. A dental component, comprising:

a porous layer that forms an inner layer of the dental component; and

a non-porous layer that forms an outer layer of the dental component.

72. The dental component of clause 71, wherein the dental component has a thickness of about 0.5 to about 5 mm.

73. The dental component of clause 71 or 72, wherein the porous layer has a thickness of about 10 to about 200 microns.

74. The dental component any one of clauses 71-73, wherein each pore of the porous layer is about 2 to about 10 microns.

75. The dental component any one of clauses 71-74, wherein the pores are about 10% to about 70% of the surface area of the porous layer.

76. The dental component any one of clauses 71-75, wherein the porous layer has a surface roughness having an absolute depth profile of about 10 to about 1000 microns.

77. The dental component any one of clauses 71-76, wherein the dental component is formed of a resin, a ceramic, a metal, a metal alloy, or a combination thereof.

78. The dental component of clause 77, wherein the resin is a polymer.

79. The dental component of clause 77, wherein the ceramic comprises zirconia, alumina, glass, or a combination thereof.

80. The dental component of clause 77, wherein the metal or metal alloy comprises titanium gold, cobalt, chromium, palladium, or combinations thereof.

81. The dental component any one of clauses 71-80, wherein the dental component is formed of a polymeric material comprising filler.

82. The dental component any one of clauses 71-81, wherein the dental component is configured to be cemented to a tooth.

83. The dental component any one of clauses 71-82, wherein the dental component is a dental restoration, an implant, or an abutment.

84. The dental component any one of clauses 71-83, wherein the dental restoration is a crown, an inlay, a veneer, or an onlay.

85. The dental component any one of clauses 71-84, wherein the dental restoration is a crown.

86. A dental implant comprising:

an abutment comprising a multilayer material and configured to receive a dental crown;

a post located spaced apart from the abutment, the post arranged to couple the abutment to a jaw bone; and

wherein the multilayer material of the abutment comprises a porous material configured to contact soft tissue of a patient.

87. The dental implant of clause 86, wherein the multilayer material comprises an outer layer formed to include a plurality of pores and an inner layer configured to secure and reinforce the outer layer.

88. The dental implant of clause 86 or 87, wherein the inner layer is formed of a solid layer.

89. The dental implant of any one of clauses 86-88, wherein the inner layer secures the abutment to the post.

90. The dental implant of any one of clauses 86-89, wherein the outer layer has a thickness of about 100 about 300 microns.

91. The dental implant of any one of clauses 86-90, wherein the pores are sized to attract and receive oral mucosal cells.

92. The dental implant of any one of clauses 86-91, wherein the pores are placed to create strong adhesion and interlocking with oral mucosal cells such as epithelial cells.

93. The dental implant of any one of clauses 86-92, wherein the outer layer is about 20% to about 70% porous.

94. The dental implant of any one of clauses 86-93, wherein each pore is about 50 microns to about 350 microns long.

95. The dental implant of any one of clauses 86-94, wherein the porous material creates a surface roughness having an absolute depth profile of about 1 to about 100 microns.

96. The dental implant of any one of clauses 86-88, wherein the abutment is formed of a metal a polymeric composition, or a ceramic.

97. The dental implant of clause 96, wherein the polymeric composition comprises a polymeric material and a filler.

98. The dental implant of clause 97, wherein the filler comprises a ceramic.

99. The dental implant of clause 98, wherein the ceramic comprises zirconia or a glass ceramic.

100. The dental implant of clause 96, wherein the polymeric composition comprises PEEK or a dental composite resin.

101. The dental implant of any one of clauses 86-100, wherein the crown is formed of a metal, a ceramic, or a polymeric composition.

102. The dental implant of clause 101, wherein the ceramic comprises zirconia or a glass ceramic.

103. The dental implant of clause 101, wherein the polymeric composition comprises PEEK or a dental composite resin.

104. The dental implant of any one of clauses 86-103, wherein the post is formed of a metal, a ceramic, or a polymeric composition.

105. The dental implant of clause 104, wherein the ceramic comprises zirconia or a glass ceramic.

106. The dental implant of clause 104, wherein the polymeric composition comprises PEEK or a dental composite resin.

107. A post for a tooth root of a tooth comprising:

a shaft formed of a lattice material, the shaft being configured to extend through a prepared tooth to couple a dental crown to the prepared tooth,

wherein the post is formed of a metal, a ceramic, a glass ceramic, or a polymeric material.

108. The post of clause 107, wherein the shaft does not comprise a solid core.

109. The post of clause 107 or 108, wherein the polymeric material comprises fiber reinforcement.

110. The post of any one of clauses 107-109, wherein the post is formed of a tooth-colored material.

111. The post of any one of clauses 107-110, wherein the post comprises zirconia, alumina, or a combination thereof.

112. The post of any one of clauses 107-111, wherein the tooth is prepared by a root canal.

113. The post of any one of clauses 107-112, wherein the tooth has compromised retention.

114. The post of any one of clauses 107-113, wherein the post is configured to be cemented to the tooth.

115. The post of any one of clauses 107-114, wherein the post is cemented with a dental cement or an adhesive resin cement.

Additional features of the present disclosure will become apparent to those skilled in the art upon consideration of illustrative embodiments exemplifying the best mode of carrying out the disclosure as presently perceived.

### BRIEF DESCRIPTION OF THE DRAWINGS

FIG. 1 shows from left to right a multilayer crown on a die and a monolayer crown on a die.

FIG. 2 is a sectional view of the multilayer crown from FIG. 1.

FIG. 3 is a sectional view of a multilayer crown having a porous inner layer.

FIG. 4 is a magnified view of a portion of FIG. 3, showing the porous structure of the inner layer.

FIG. 5 is an elevation view of a dental implant including a porous abutment.

FIG. 6 is a sectional view of a porous post for a dental implant.

### DETAILED DESCRIPTION

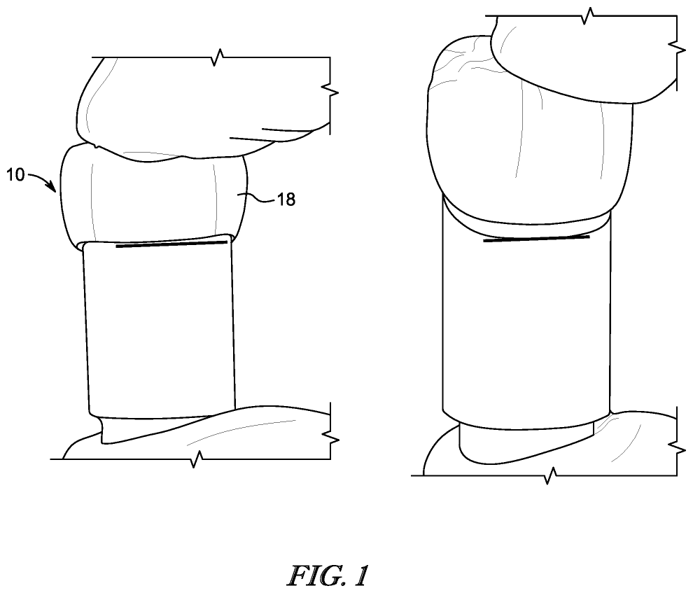

Dental restorations, such as prefabricated dental crowns, may require chair-side adjustments to ensure an acceptable fit to a patient's tooth. Rigid dental crowns may be unable to accommodate much error between the rigid crown itself and the patient's tooth. In contrast, a multilayer crown 10 is configured to compensate for the differences between a patient's tooth and the multilayer crown 10 itself. As shown in FIG. 1, a rigid dental crown (FIG. 1, right) is unable to properly seat on a mis-sized tooth die so that the die fills the interior space (denoted by the line on the die). In contrast, the multilayer crown 10 (FIG. 1, left) is capable of accepting the mis-sized die. In some illustrative embodiments, the multilayer crown 10 is flexible. In some embodiments, the multilayer crown 10 can be compressed manually and return to its original shape when the compressive force is removed.

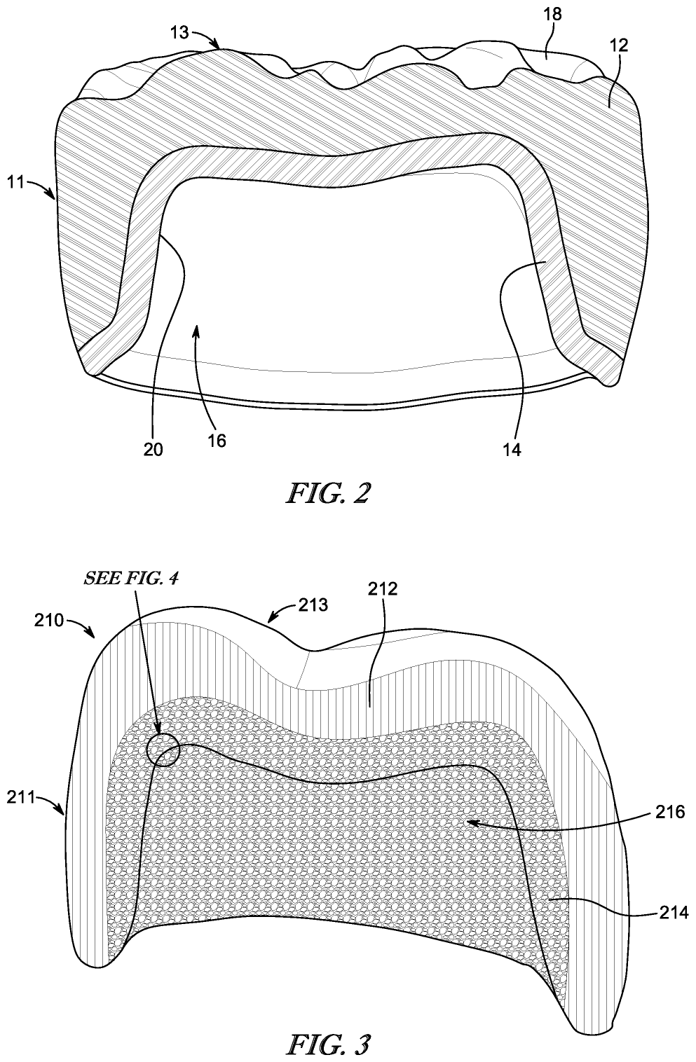

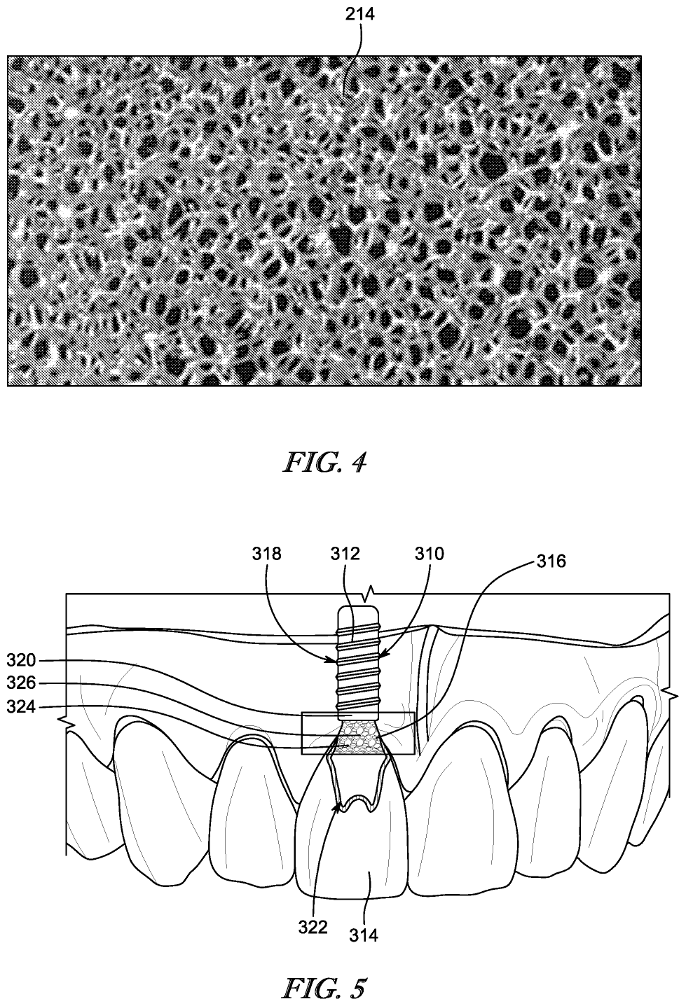

Although the multilayer crown 10 is specifically embodied in FIGS. 1 and 2, this disclosure applies equally to other dental restorations such as inlays, veneers, onlays, and other dental components that are known in the art but now shown.

Multilayer crown 10 includes an outer layer 12, an inner layer 14, and an interior region 16, as shown in FIG. 2. Inner layer 14 is located spaced apart from the outer layer 12, as shown in FIG. 2. The outer layer 12 is arranged to form a surface 18 that interacts with a patient's mouth. The interior region 16 is sized to receive a patient's tooth. The inner layer 14 has a surface 20 that is arranged to form the interior region 16. The outer layer 12, the inner layer 14, and the interior region 16 cooperate to couple the multilayer crown 10 to the patient's tooth and protect the patient's tooth. In illustrative embodiments, the multilayer crown 10 consists of two layers. Illustratively, the inner layer 14 directly contacts the outer layer 12, the inner layer 14 forms an external surface of the multilayer crown 10 and the outer layer 12 forms the opposite external surface.

In illustrative embodiments, the multilayer crown 10 includes a sidewall 11 and a top 13, as shown in FIG. 2. The sidewall 11 is arranged to surround the sides of a patient's tooth. The top 13 is arranged to form the top of the multilayer crown 10. The sidewall 11 and the top 13 cooperate to enclose the patient's tooth inside the interior region 16.

In illustrative embodiments, each of the outer layer 12 and the inner layer 14 are formed of a material that has a hardness. Illustratively, the hardness of the outer layer 12 is greater than the hardness of the inner layer 14. In illustrative embodiments, each of the outer layer 12 and the inner layer 14 are formed of a material that has an elasticity. Illustratively, the elasticity of the outer layer 12 is less than the elasticity of the inner layer 14. In some embodiments, the inner layer 14 has a hardness that is lower than a hardness for the outer layer 12 and the inner layer 14 has an elasticity that is higher than the elasticity of the outer layer 12.

In some aspects, having a softer inner layer 14 allows the multilayer crown 10 to adapt to the shape of the underlying tooth. Illustratively, this may allow the multilayer crown 10 to compensate for differences between the prepared tooth and the interior space. In some embodiments, the multilayer crown 10 can compensate up to about 50%, up to about 40%, up to about 30%, up to about 25%, or up to about 15% difference between the volume of the interior region 16 and the patient's tooth. In some embodiments, the multilayer crown 10 can compensate about 50%, about 40%, about 30%, about 25%, about 20%, about 15%, about 10%, about 5% or about 1% difference between the volume of the interior region 16 and the patient's tooth.

The inner layer 14 forms a particular percentage of the volume of the multilayer crown 10. In some aspects, the inner layer 14 is about 10% to about 75%, about 10% to about 70%, about 10% to about 60%, about 10% to about 50% of the total volume of the multilayer crown 10. In some embodiments, the remaining volume of the multilayer crown 10 is the outer layer 12.

The inner layer 14 is formed of a composition. In some illustrative embodiments, the inner layer 14 is formed of a composition that has been cured, extruded, or laminated. Illustrative curing techniques include UV, heat, and those otherwise known in the art.

In some aspects, the composition comprises a polymeric material. In some aspects, the composition is a composite. In some embodiments, the composition comprises a polymeric material and a filler. Illustrative polymeric materials include triethylene glycol dimethacrylate (TEGDMA), polymethylmethacrylate (PMMA), urethane dimethylacrylate (UDMA), polyglycidyl methacrylate (Bis-GMA), or mixtures thereof.

Illustrative fillers include glass fillers, ceramics, combinations thereof, or those otherwise known in the art. Illustrative glass filers include barium glass. Illustrative ceramics may comprise zirconia or alumina. In some embodiments, the composition of the inner layer 14 includes less filler than the composition for the outer layer 12. In some embodiments, composition comprises about 1% to about 60% filler, about 1% to about 50%, about 1% to about 40%, about 1% to about 35%, or about 10% to about 40% filler. The amount of filler may be adjusted depending on the type of filler and the expected viscosity.

In some aspects, the inner layer 14 has a flexural modulus. Flexural modulus can be measured through a three-point bending, a four point bending, or a bi-axial flexural test. Illustratively, flexural modulus can be measured according to the ISO standards. For example, ISO Standards 6872/2015, 178, 1567, or 4049. In some embodiments, the inner layer 14 has a flexural modulus less than the flexural modulus of the outer layer 12. In some embodiments, the inner layer 14 has a flexural modulus of less than about 2,500, less than about 2,400, less than about 2,300, less than about 2,200, or less than about 2,100 MPa. In some embodiments, the flexural modulus for the inner layer 14 is about 1,500 MPa to about 2,500 MPa.

In some aspects, the inner layer 14 has a flexural strength as measured by according to the ISO standards. For example, ISO Standards 6872/2015, 178, 1567, or 4049 may be used. In some embodiments, the inner layer 14 has a flexural strength less than the flexural strength of the outer layer 12. In some embodiments, the inner layer 14 has a flexural strength of less than about 60, less than about 55, less than about 50, or less than about 45 MPa. In some embodiments, the flexural strength for the inner layer 14 is about 35 to about 150 MPa.

In some aspects, the inner layer 14 has a modulus of elasticity as measured by the appropriate ISO Standard. In some embodiments, the inner layer 14 has a modulus of elasticity less than the modulus of elasticity of the outer layer 12. In some embodiments, the inner layer 14 has a modulus of elasticity of less than about 2,500, less than about 2,400, less than about 2,300, or less than about 2,000 MPa. In some embodiments, the modulus of elasticity for the inner layer 14 is about 1,500 to about 2,500, about 1,500 to about 2,400, about 1,500 to about 2,300, or about 1,500 to about 2,000 MPa.

In some aspects, the inner layer 14 has a elongation at break that can be measured according to appropriate ISO Standards. For example, ISO Standard 1421 may be used. In some embodiments, the inner layer 14 has a elongation at break greater than the elongation at break of the outer layer 12. In some embodiments, the inner layer 14 has a elongation at break greater than about 20%, greater than about 25%, or greater than about 30%. In some embodiments, the elongation at break for the inner layer 14 is about 20% to about 40%, about 20% to about 35%, about 20% to about 30%, or about 20% to about 25%.

The outer layer 12 forms a particular percentage of the volume of the multilayer crown 10. In some aspects, the outer layer 12 is about 10% to about 75%, about 10% to about 70%, about 10% to about 60%, about 10% to about 50% of the total volume of the multilayer crown 10. In some embodiments, the remaining volume of the multilayer crown 10 is the inner layer 14.

The outer layer 12 is formed of a composition. In some aspects, the composition is a composite. In some illustrative embodiments, the outer layer 12 is formed of a composition that has been cured, extruded, or laminated. Illustrative curing techniques include UV, heat, and those otherwise known in the art.

In some aspects, the composition comprises a polymeric material. In some aspects, the composition of the outer layer 12 is formed of the same materials as inner layer 14 but in different relative amounts of each component. In some embodiments, the composition comprises a polymeric material and a filler. Illustrative polymeric materials include triethylene glycol dimethacrylate (TEGDMA), polymethylmethacrylate (PMMA), urethane dimethylacrylate (UDMA), polyglycidyl methacrylate (Bis-GMA), or mixtures thereof.

Illustrative fillers include glass fillers, ceramics, combinations thereof, or those otherwise known in the art. Illustrative glass filers include barium glass. Illustrative ceramics may comprise zirconia or alumina. In some embodiments, the composition of the inner layer 14 includes less filler than the composition for the outer layer 12. In some embodiments, the outer layer 12 comprises less than about 80% filler. In some embodiments, composition comprises about 30% to about 80% filler, about 40% to about 80%, about 50% to about 80%, or about 50% to about 70% filler. The amount of filler may be adjusted depending on the type of filler and the expected viscosity.

In some aspects, the outer layer 12 has a flexural modulus. Flexural modulus can be measured through a three-point bending, a four point bending, or a bi-axial flexural test. Illustratively, a three point bending test can be performed according to ISO Standard 6872/2015 or by ISO Standard 178. For example, ISO Standards 6872/2015, 178, 1567, or 4049 may be used In some embodiments, the outer layer 12 has a flexural modulus greater than the flexural modulus of the inner layer 14. In some embodiments, the outer layer 12 has a flexural modulus of greater than about 2,500, greater than about 3,000, greater than about 3,500, greater than about 4,000, or greater than about 5,000 MPa. In some embodiments, the flexural modulus for the outer layer 12 is about 2,500 MPa to about 6,000 MPa.

In some aspects, the outer layer 12 has a flexural strength as measured by according to the ISO Standards. For example, ISO Standards 6872/2015, 178, 1567, or 4049 may be used. In some embodiments, the outer layer 12 has a flexural strength greater than the flexural strength of the inner layer 14. In some embodiments, the outer layer 12 has a flexural strength of greater than about 65, greater than about 70, greater than about 75, or greater than about 80 MPa. In some embodiments, the flexural strength for the outer layer 12 is about 65 to about 150 MPa.

In some aspects, the outer layer 12 has a modulus of elasticity as measured by the appropriate ISO Standard. In some embodiments, the outer layer 12 has a modulus of elasticity greater than the modulus of elasticity of the outer layer 12. In some embodiments, the outer layer 12 has a modulus of elasticity of greater than about 2,500, greater than about 3,000, greater than about 3,500, or greater than about 4,000 MPa. In some embodiments, the modulus of elasticity for the outer layer 12 is about 2,500 to about 6,000, about 3,000 to about 6,000, or about 3,500 to about 6,000 MPa.

In some aspects, the outer layer 12 has a elongation at break that can be measured according to the appropriate ISO Standards. For example ISO Standard 1421 may be used. In some embodiments, the outer layer 12 has a elongation at break less than the elongation at break of the outer layer 12. In some embodiments, the outer layer 12 has a elongation at break less than about 20%, less than about 15%, or less than about 10%. In some embodiments, the elongation at break for the outer layer 12 is about 5% to about 20%.

The multilayer crown 10 can be formed through a variety of techniques known in the art. In some embodiments, the multilayer crown 10 is formed through additive manufacturing. Illustrative additive manufacturing techniques include jet printing, stereolithography, digital light processing, extrusion, coextrusion, lamination, and combinations of those techniques. Additional additive manufacturing techniques are known to those skilled in the art.

In some embodiments, the multilayer crown 10 is formed through milling. For example a pre-made block of material can be milled to form the multilayer crown 10.

In some embodiments, a formulation is used in an additive manufacturing process to form an outer layer. Illustratively, the formulation may be printed, jet printed, extruded, or laminated. The formulation may then be cured by heat, UV, or other curing techniques known in the art. In some embodiments, the formulation is cured to form outer layer 12.

In some embodiments, a formulation is used in an additive manufacturing process to form an outer layer. Illustratively, the formulation may be printed, jet printed, extruded, or laminated. The formulation may then be cured by heat, UV, or other curing techniques known in the art. In some embodiments, the formulation is cured to form inner layer 14.

In some embodiments, a first formulation is used in an additive manufacturing process to form the outer layer 12. In some embodiments, a second formulation is used in an additive manufacturing process to form the inner layer 14. Illustratively, the first formulation may be printed, jet printed, extruded, or laminated alongside a second formulation. Each layer may then be cured individually or together to form the outer layer 12 and the inner layer 14.

In an alternative embodiment, a pre-made block is formed having two materials. The two materials may be arranged so that a multilayer crown 10 can be formed through a milling process.

In some embodiments, a patient's prepared tooth is digitized. The digitized tooth can then serve as the basis for preparing the multilayer crown 10.

In another embodiment, a multilayer crown 210 includes an outer layer 212, an inner layer 214, and an interior space 216, as shown in FIG. 3. Illustratively, the inner layer 214 is formed of a porous material. The porous material is configured to adhere to a patient's prepared tooth. In some embodiments, the multilayer crown 210 is formed of ceramic materials. Although the following describes a multilayer crown 210, the description applies equally to dental restorations, implants and abutments.

In illustrative embodiments, the multilayer crown 210 includes a sidewall 211 and a top 213, as shown in FIG. 3. The sidewall 211 is arranged to surround the sides of a patient's tooth. The top 213 is arranged to form the top of the multilayer crown 210. The sidewall 211 and the top 213 cooperate to enclose the patient's tooth inside the interior space 216. In illustrative embodiments, the sidewall 211, the top 213, or both are about 0.5 to about 5 mm or at least mm thick.

In illustrative embodiments, the inner layer 214 is formed of a porous material. In some aspects, the outer layer 212 is formed of a non-porous material. Illustratively, the porous material of the inner layer 214 may improve cement adhesion of the multilayer crown 210. In some embodiments, the pore size may be about 2 microns to about 10 microns. In some embodiments, the pores cover about 10% to about 70% of the surface area of the inner layer 214. In some embodiments, the porous structure creates a surface roughness that has an absolute depth profile or about 10 to about 1,000 microns.

In some aspects, the multilayer crown 210 is formed of composition such as a resin, a ceramic, a metal, a metal alloy, or a combination thereof. In some aspects, the composition is a composite. Illustrative resins include polymeric materials as those described herein, for example with reference to multilayer crown 10. Illustrative ceramics include those comprising zirconia, alumina, glass, combinations thereof, or those described herein with reference to multilayer crown 10. Illustrative metals or metal alloys may comprise titanium gold, cobalt, chromium, palladium, and combinations thereof. In some illustrative embodiments, the inner layer 214 and the outer layer 212 are formed of the same composition. In other illustrative embodiments, the inner layer 214 and the outer layer 212 are formed of a different composition.

The inner layer 14 forms a particular percentage of the volume of the multilayer crown 10. In some aspects, the inner layer 14 is about 10% to about 75%, about 10% to about 70%, about 10% to about 60%, about 10% to about 50% of the total volume of the multilayer crown 10. In some embodiments, the remaining volume of the multilayer crown 10 is the outer layer 12.

The outer layer 212 forms a particular percentage of the volume of the multilayer crown 10. In some aspects, the outer layer 212 is about 10% to about 75%, about 10% to about 70%, about 10% to about 60%, about 10% to about 50% of the total volume of the multilayer crown 210. In some embodiments, the remaining volume of the multilayer crown 210 is the inner layer 214.

In another embodiment, a dental implant 310 comprises an post 312, a crown 314, and an abutment 316, as shown in FIG. 5. The post 312 is configured to secure the dental implant 310 to a patient. The crown 314 is arranged to interact with the patient's other teeth and mouth. The abutment 316 extends between and interconnects the crown 314 and the post 312. The abutment 316 comprises a porous material that is arranged to contact the soft tissue of a patient when the dental implant 310 is installed in the patient.

In illustrative embodiments, the post 312 includes threads 318 that cooperate to secure the dental implant 310 to bone and an abutment receiver 320, as shown FIG. 5. The abutment receiver 320 is configured to receive the abutment 316. The post 312 may be formed of any material known in the dental arts for dental implants.

The crown 314 is spaced-apart from the post 312. The crown 314 is formed to resemble a patient's natural tooth. Illustratively, the crown 314 is formed of a ceramic material or any of the other materials described herein for multilayer crowns 10, 210. The crown 314 includes an abutment receiver 322 that is configured to couple the crown 314 to the abutment 316, as shown in FIG. 5.

The abutment 316 is located between post 312 and the crown 314, as shown in FIG. 5. The abutment 316 coupled the crown 314 to the post 312. The abutment 316 comprises an outer surface 324 that contacts the soft tissue, for example the gums, of a patient when implanted.

In some aspects, the abutment 316 includes a portion that is arranged to contact the soft tissue of a patient's mouth and a portion that is arranged to receive the crown 314. The abutment 316 may be formed of a material that is porous. In some embodiments, the entirety of the abutment 316 is porous or only a portion of the abutment 316 contacting the soft tissue is porous, as shown in FIG. 5.

In some aspects, the abutment 316 comprises a multilayer material. The multilayer material includes an inner layer and an outer layer. The inner layer is configured to secure and reinforce the outer layer. In some embodiments, the inner layer is formed of a solid material. The outer layer is arranged to contact the patient's soft tissue. In some embodiments, the outer layer is formed of the porous material.

In illustrative embodiments, the abutment 316 comprises an outer layer 326 that is formed of a porous material. In some embodiments, the outer layer 326 is about 100 to about 500 microns thick as measured from the outer surface 324. In some embodiments, the outer layer 326 is about 150 to about 450 microns or about 150 to about 400 microns thick. In some embodiments, the outer layer may have a thickness of about 100 to about 300 microns.

Illustratively, the porous material comprises a plurality of pores. The pores may be sized so that cells of the neighboring tissue may grow therein. In some embodiments, the outer layer is about 20% to about 70% porous or about 20% to about 50% porous. Illustratively, each pore may be sized to receive and/or attract an oral mucosal cell. In some embodiments, each pore is about 50 microns to about 350 microns, about 50 microns to about 300 microns, about 100 microns to about 300 microns long, or about 100 microns to about 200 microns. In some embodiments, the porous structure creates a surface roughness. In some embodiments, the surface roughness has an absolute depth profile to about 1 to about 100 microns.

The abutment 316 or the components thereof may be formed of zirconia, glass ceramic, polymeric materials, combinations thereof, or any other material for dental applications. In some aspects, the abutment 316 is formed of a metal, a ceramic, a polymeric material, or a dental composite. Illustrative ceramics may comprise zirconia or be a glass ceramic. Illustrative polymeric materials include those described herein and PEEK.

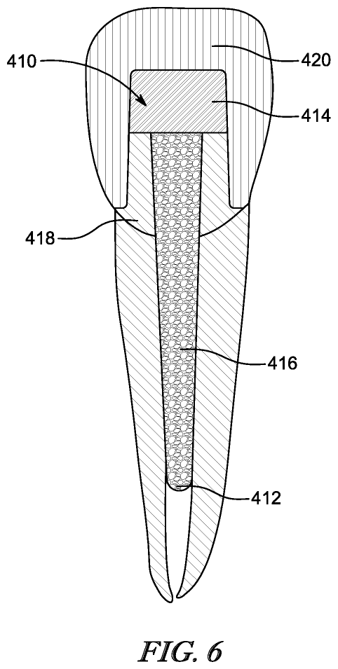

In another embodiment, a dental post 410 for a tooth root comprises a distal end 412, a crown receiver 414, and a shaft 416, as shown in FIG. 6. The dental post 410 comprises a lattice material that is configured to bond to the tooth 418 of the patient. In some aspects, the tooth has been prepared through a root canal. The shaft 416 is extends down through a prepared tooth to secure the dental post 410 to the patient's tooth. The crown receiver 414 secures the crown 420 to the dental post 410.

Illustratively, the lattice of the shaft 416 comprises a plurality of pores. The plurality of pores cooperate to couple the dental post 410 to the patient. In some aspects, the shaft 416 does not include a solid core.

In some aspects, the shaft 416 is secured to the prepared tooth 418. Illustratively, the shaft 416 may be cemented through a dental cement or an adhesive resin.

In some aspects, the dental post 410 is formed of a composition. In some embodiments, the post 410 is formed of a metal, a ceramic, a glass ceramic, a polymeric material, fiber reinforced polymers, or a combination thereof. In some embodiments, the dental post 410 may be formed of tooth colored materials. In some embodiments, the composition comprises zirconia, alumina, or a combination thereof.

### EXAMPLES

### Example 1

The purpose of this example was to assess the feasibility of additively manufacturing a dental crown with a two-layer design using a material jetting printer.

A mandibular first molar denture tooth from a dental typodont (Basic study model; Kayo) was digitized using a structured light scanner (S300 ARTI scanner; Zirkonzhon). The standard tessellation language (STL₁) file was obtained and imported into a computer aided design (CAD) software program (Geomagic Freeform; 3D Systems). A tooth preparation for a full coverage crown with 1.5 mm occlusal reduction, 1.5 mm axial reduction, a 1.5 mm circumferential chamfer margin, and a total occlusal convergence of 10 degrees was designed using a software program (Geomagic Freeform; 3D Systems). Subsequently, the STL₂ file was exported and used to manufacture a titanium grade 5 (Starbond Ti5 Disc; Scheftner) milled (Arum 5x-200, Doowon USA, Inc.) tooth preparation die.

The metal die was digitized using a laboratory scanner (E4 Scanner; 3Shape) following the manufacturer's recommendations. The scanner was previously calibrated following the manufacturer's protocol. A STL₃ file was obtained and imported into a CAD software program (Dental System; 3Shape) and an anatomically contoured crown for a first mandibular molar was obtained with a uniform thickness of 1.5 mm. The virtual design file (STL₄ file) was exported.

The STL₄ file was imported into the CAD software program (Geomagic Freeform; 3D Systems). Subsequently, two virtual crown designs were obtained, namely monolayer (ML group) and 2-layer (2L group) designs (Table 1).

TABLE 1

| Group | Material | Layer |
|---|---|---|
| Monolayer | Rigur RGD450; Stratasys | Entire crown thickness |
| 2L group | VeroClear; | Intaglio layer (25% volume of the |
| | Stratasys (white) | crown design) |
| | Rigur RGD450; | Exterior or superficial layer (75% |
| | Stratasys (clear) | volume of the crown design) |

For the monolayer crown design, the digital crown design was manufactured with a hard polymer (Rigur RGD450; Stratasys) using a material jetting printer (Connex3 Object260; Stratasys) following the manufacturer recommendations.

For the two-layer crown design, the digital crown was splinted into 2 parts: the intaglio of the crown that represented 25% of the total crown volume and the exterior that represented the 75% remaining crown volume (see FIG. 2). The virtual design was imported into the printer software program (GrabCAD; Stratasys) and manufactured using two different materials. The intaglio part was manufactured using a resilient polymer (Vero; Stratasys) while the exterior part was manufactured with a hard polymer (Rigur RGD450; Stratasys) using a material jetting printer (Connex3 Object260; Stratasys) following the manufacturer recommendations.

The crown integrity and marginal discrepancy of the specimens of ML and 2L groups were visually evaluated on the milled tooth preparation die.

The monolayer and two-layer crown designs were manufactured using a material jetting printer. The crowns in all groups manufactured with acceptable anatomical shape and show structural integrity with no visible defects. Crowns in all groups fit the titanium tooth preparation die without any adjustment and the visual examination determined that all the specimens obtained an acceptable marginal discrepancy. Longitudinal sectioned crowns showed acceptable internal integrity in all groups with visible transition of layers in the two-layer crown design.

The monolayer and two-layer additively manufactured crowns were obtained using a material jetting printer. The present example demonstrated the feasibility of designing a multi-layer dental restoration and manufacturing a multi-material dental crown using a material jetting printer.

In an additively manufactured bio-inspired restoration, the crown should resemble the structure and mechanical properties of the natural dental tissues. In this example, although the two-layer designs might not replicate the mechanical properties of enamel and dentin; manufacturing multi-materials to potentially represent enamel and dentin in x-, y-, and z-axes could be considered the first attempt in replacing missing tooth tissues (structure) with a bio inspired concept using an additively manufacturing technology. Due to the limited materials currently available to be processed using the material jetting printer selected, the materials used to manufacture the specimens were selected based on the differences in their mechanical properties to represent enamel and dentin. The hard material aimed to mimic the mechanical properties of dental enamel, while the soft material intended to replicate the dentine properties (Table 2).

TABLE 2

| Property | Rigur RGD450 | VeroClear |
|---|---|---|
| Tensile strength | 40-45 MPa | 40-55 MPa |
| Elongation at break | 20-35% | 5-20% |
| Modulus of elasticity | 1700-2100 MPa | 2200-3000 MPa |
| Flexural strength | 52-59 MPa | 70-85 MPa |
| Flexural modulus | 1500-1700 MPa | 2000-2500 MPa |
| Color | White | Clear |

The marginal and internal discrepancies evaluation of the specimens demonstrated clinically acceptable marginal and internal discrepancies; however, further studies are needed to assess the internal and marginal discrepancies of the AM restorations manufactured with two-layer designs. Additionally, the mechanical properties of the AM two-layer design should be analyzed.

Technology improvement and new materials development are fundamental elements for the implementation of manufacturing standards as well as the clinical development of the AM dental applications. The continuous developments of the polymers and material jetting technology facilitate the progress of additively manufactured bio-inspired dental restorations. The optimization of the printer nozzle, printing parameters, or printing accuracy may facilitate the future improvement of the concept described on the present example.

### Example 2

Subtractive computer-aided manufacturing (CAM) technologies are commonly used to fabricate ceramic fixed dental prostheses (FDPs). CAM technologies typically refer to a computer numerically controlled (CNC) system that controls power-driven machine tools. Under the direction of computer software, these tools mechanically remove material from a ceramic block to carve the desired prostheses. Although subtractive technologies are considered the gold standard for the fabrication of FPDs, they also present a number of manufacturing limitations, including the amount of material wasted, the milling tool's short lifetime, and the space limitations imposed by the size of the milling burs and the axis of the CNC machine, limiting the access to smaller areas of the milling block.

Additive manufacturing (AM) technologies that can be used for processing zirconia include vat photo-polymerization, material extrusion, and direct inkjet printing. In the vat-polymerization procedures, such as stereolithography (SLA) technology, a liquid resin is mixed in a ceramic suspension and selectively solidified through controlled photopolymerization. Consequently, green parts with different shapes can be fabricated by using a ceramic suspension that is a mixture of ceramic powders and photosensitive resin. Postprocessing of the fabricated green parts is necessary to eliminate organic materials in photosensitive resin and fuse the ceramic particles together to obtain dense ceramic components.

AM technologies provide a method that allows the manufacturing of a porous product which can substantially influence its mechanical, physical, chemical, and biologic properties. To develop a bioinspired dental material that could imitate or regenerate complex biologic systems, restorative materials should be able to mimic enamel and dentin tissues. Considering the limitations of both conventional and subtractive manufacturing methods, AM technologies may provide a new manufacturing method that enhances the clinical performance of restorative dental materials.

Similar to milled partially sintered or nonsintered zirconia material, the AM zirconia material requires sintering procedures after printing, followed by elimination of the photosensitive resin of the AM green part. In subtractive techniques, the sintering procedure is accompanied by shrinkage of approximately 20% to 30% of the total volume of a restoration, which is compensated by an expanded digital design of the restoration. However, in AM procedures, the sintering shrinkage remains unclear.

Trueness and precision define the accuracy of a 3D printer. Trueness relates to the ability of the printer to reproduce an object as close to its virtual design as possible, whereas precision indicates the difference among objects manufactured under the same conditions.

The purpose of this in vitro study was to measure the manufacturing accuracy and volumetric changes of SLA AM zirconia specimens with porosities of 0%, 20%, and 40%. The null hypotheses were that no significant differences in the specimen dimensions (length, width, and height) would be found among the 0%-, 20%-, and 40%-porosity SLA AM zirconia specimens and that no significant difference in manufacturing volumetric changes would be found among the 0%-, 20%-, and 40%-porosity SLA AM zirconia specimens.

A digital design for a bar (25×4×3 mm) was created by using an open source software program (Blender, version 2.77a; The Blender Foundation). The standard tessellation language (STL0%) file was exported.

Three groups were created based on the porosity of the specimens: 0% porosity (0% group), 20% porosity (20% group), and 40% porosity (40% group). The STL file was used to manufacture all the specimens from a zirconia paste (3DMix ZrO₂ paste; 3DCeram Co) mixed with liquid photosensitive resin in a ceramic 3D printer (CeraMaker900; 3DCeram Co). After the AM process was completed, the specimens were cleaned by using a semiautomated cleaning station. Subsequently, the binder was removed in a furnace at 600° C. The temperature was increased to 1050° C. to facilitate removing and transferring the specimens to a sintering furnace. The sintering procedures varied among the groups to achieve different porosities. For the 0% group, the ZrO₂ was sintered in a furnace at 1400° C., and for the 20% and 40% groups, the sintering temperature varied between 1450° C. and 1225° C. The sintering details are the proprietary information of the manufacturer. No additional processing, including finishing or polishing, was performed. All the specimens of the same group were manufactured at the same time to standardize the manufacturing procedures. All the AM specimens were produced by the manufacturer (3DCeram Co).

The dimensions (length, width, and height) of all AM specimens were measured with digital calipers (Mitutoyo500-196-20 6′ Digimatic Caliper; Mitutoyo). The manufacturer of this digital caliper reports an accuracy of 0.01 mm Each measurement was performed three times, and the mean value was determined. The manufacturing volume shrinkage (%) was calculated using the digital design of the bar and the AM dimensions of the specimens.

The Shapiro-Wilk test revealed that the data were not normally distributed. Therefore, the data were analyzed by using the Kruskal-Wallis followed by pairwise Mann-Whitney U tests (a=0.05) with a statistical software pro-gram (IBM SPSS Statistics for Windows, v25; IBM Corp).

The Kruskal-Wallis test demonstrated significant differences among the groups in length, width, and height dimensions (P<0.001). The Mann-Whitney U test indicated significant differences in the pairwise comparisons of length, width, and height dimensions among the 3 groups (P<0.001). The 0% group obtained median±interquartile range values of 20.92±0.14 mm in length, 3.43±0.07 mm in width, and 2.39±0.03 mm in height; the 20% group mean values were 22.81±0.29 mm in length, 3.74±0.07 mm in width, and 2.62±0.05 mm in height; and the 40% group mean values were 25.11±0.13 mm in length, 4.14±0.08 mm in width, and 2.96±0.02 mm in height (Tables 3-5).

Table 5 provides the manufacturing volumetric changes of the specimens for each group. Significant differences were found in the manufacturing volumetric changes among the groups (P<0.001).

TABLE 3
Length, width, and height data for tested groups (mm)

| | | 0% Group | 20% Group | 40% Group |
|---|---|---|---|---|
| | | (0% | (20% | (40% |
| Dimension | Value | Porosity) | Porosity) | Porosity) |
| Length | Median ± IQR | 20.92 ± 0.14 | 22.81 ± 0.29 | 25.11 + 0.13 |
| | Percentile 25 | 20.81 | 22.63 | 25.06 |
| | Percentile 75 | 20.95 | 22.92 | 25.19 |
| Width | Median ± IQR | 3.43 ± 0.07 | 3.74 ± 0.07 | 4.14 + 0.08 |
| | Percentile 25 | 3.39 | 3.70 | 4.11 |
| | Percentile 75 | 3.46 | 3.77 | 4.19 |
| Height | Median ± IQR | 2.39 ± 0.03 | 2.62 ± 0.05 | 2.96 + 0.02 |
| | Percentile 25 | 2.37 | 2.60 | 2.95 |
| | Percentile 75 | 2.40 | 2.65 | 2.97 |

IQR, interquartile range.

TABLE 4
Trueness and precision values for tested groups (mm)

| | 0% Group | | 20% Group | | 40% Group | |
|---|---|---|---|---|---|---|
| | (0% Porosity | | (20% Porosity) | | (40% Porosity) | |
| Dimension | Trueness | Precision | Trueness | Precision | Trueness | Precision |
| Length (x-axis) | 4.08 | 0.14 | 2.19 | 0.29 | 0.11 | 0.13 |
| Width (y-axis) | 0.57 | 0.07 | 0.26 | 0.07 | 0.14 | 0.08 |
| Height (z-axis) | 0.62 | 0.03 | 0.38 | 0.05 | 0.04 | 0.02 |

TABLE 5
Manufacturing volume changes for tested groups (%)

| | 0% Group | 20% Group | 40% Group |
|---|---|---|---|
| | (0% Porosity) | (20% Porosity) | (40% Porosity) |
| Dimension | Median ± IQR | Median ± IQR | Median ± IQR |
| Length | −16.32 ± 0.57 | −8.76 ± 1.16 | +0.44 ± 0.52 |
| Width | −14.25 ± 1.75 | −6.5 ± 1.75 | +3.5 ± 2.00 |
| Height | −20.33 ± 1.00 | −12.67 ± 1.67 | −1.33 ± 0.66 |

IQR, interquartile range. Negative values indicate manufacturing shrinkage. Positive values indicate larger volume than digital design of specimens.

Significantly different manufacturing accuracies and volumetric changes were found among the groups. Furthermore, an uneven manufacturing volume change in the x-, y-, and z-axis was observed, and none of the groups tested were able to manufacture a perfect match compared with the virtual design of the specimens. Therefore, both null hypotheses were rejected.

Processing zirconia with SLA AM technologies represents a challenge because of the difficulty in controlling the volumetric changes that occur during the fabricating procedures, including the elimination of the photosensitive resin after printing the object and the sintering procedures. Limited information is available regarding the manufacturing volumetric changes that occur when processing zirconia with an SLA AM ceramic printer. However, SLA-manufactured alumina specimens have been reported to undergo anisotropic sintering shrinkage in contrast with their subtractive manufactured counterparts, which undergo homogenous shrinkage. Revilla-Leon et al. evaluated the marginal and internal gap of milled and SLA AM zirconia crowns. On the SLA AM groups, two different crown designs were tested, namely an anatomic contoured crown and a splinted crown that represented the dentin replacement of the crown. It was reported a clinically acceptable marginal and internal gap only on the splinted crown group, which could be explained by the smaller thickness of the zirconia material while maintaining the same manufacturing workflow. However, resolving conclusions from one study is complicated, and the manufacturing procedure per se was not evaluated.

This example analyzed the manufacturing volumetric changes obtained in an SLA AM zirconia printing procedure with different porosities namely 0% porosity or 100% density, 20% porosity, and 40% porosity. Based on the results of the present study, none of the groups tested were able to perfectly replicate the virtual design of the specimens. The same dimensions on the virtual design of the bar specimens were used to fabricate all the specimens. The 40%-porosity group obtained the closest dimensions to the virtual design, being the group with the lowest volumetric changes after manufacturing. Furthermore, volumetric changes observed in all directions were nonuniform compared with the virtual design of the specimen, which adversely affected the manufacturing accuracy of the desired object.

A photopolymerizable ceramic suspension is used in the ceramic SLA AM technology. The ceramic particle size, density, and refractive index of the powder, as well as the composition and proportion of the photopolymerizable solutions of the slurry used in an SLA printer will influence the sintering procedure, microstructure development, and mechanical properties of the AM ceramic part. In the present example, all of the specimens were fabricated by the manufacturer. The composition of the zirconia slurry and sintering procedures were not disclosed by the manufacturers to protect their proprietary information.

Limitations of the present study related to the different manufacturing technology, photopolymerizable ceramic suspension, printing parameters, sintering procedures, and postprocessing procedures. Moreover, different ceramic slurry mixtures may result in different results to those obtained in the present study.

Based on the findings of this in vitro study, the following conclusions were drawn:

1. The 40%-porosity AM zirconia had the highest manufacturing accuracy and the lowest manufacturing volume change, followed by the 20%-porosity and the 0%-porosity groups.

2. An uneven manufacturing volume change in the x-, y-, and z-axis was observed.

3. None of the groups tested were able to perfectly replicate the virtual design of the specimens.

1. A multilayer dental restoration, comprising
an outer layer formed of a first polymeric material,
an inner layer formed of a second polymeric material that is different from the first polymeric material, the inner layer arranged to contact a tooth so that the inner layer is located between the outer layer and the tooth,
wherein the inner layer has a hardness that is lower than a hardness for the outer layer and the inner layer has an elasticity that is higher than an elasticity of the outer layer so that the multilayer dental restoration can compensate up to about 25% of manufacturing error of the multilayer dental restoration.

2. The multilayer dental restoration of claim 1, wherein the inner layer has a flexural modulus of about 1500 to 2500 MPa.

3. The multilayer dental restoration of claim 1, wherein the outer layer has a flexural modulus of about 2500 MPa to about 6000 MPa.

4. The multilayer dental restoration of claim 1, wherein the inner layer has a flexural strength of about 35 to 50 MPa.

5. The multilayer dental restoration of claim 1, wherein the outer layer has a flexural strength of about 65 MPa to about 150 MPa.

6. The multilayer dental restoration of claim 1, wherein the inner layer has a modulus of elasticity of about 1,500 MPa to 2,500 MPa.

7. The multilayer dental restoration of claim 1, wherein the outer layer has a modulus of elasticity of about 2,500 MPa to about 6,000 MPa.

8. The multilayer dental restoration of claim 1, wherein the inner layer has an elongation at break of more than 20%.

9. The multilayer dental restoration of claim 1, wherein the outer layer has an elongation of break of about 5% to about 20%.

10. The multilayer dental restoration of claim 1, wherein the inner layer is about 10% to about 75% by volume of the multilayer dental restoration.

11. The multilayer dental restoration of claim 1, wherein the first polymeric material is a composite.

12. The multilayer dental restoration of claim 1, wherein the first polymeric material comprises triethylene glycol dimethacrylate (TEGDMA), Bis-GMA, Urethane dimethacrylate, or poly methylmethacrylate.

13. The multilayer dental restoration of claim 1, wherein the first polymeric material comprises a filler.

14.-17. (canceled)

18. The multilayer dental restoration of claim 1, wherein the second polymeric material is a composite.

19. The multilayer dental restoration of claim 1, wherein the second polymeric material comprises triethylene glycol dimethacrylate (TEGDMA), Bis-GMA, Urethane dimethacrylate, or poly methylmethacrylate.

20. The multilayer dental restoration of claim 1, wherein the second polymeric material comprises a filler.

21.-31. (canceled)

32. A method for forming a multilayer dental restoration, comprising:
providing a first material to form a first layer;
providing a second material that has different properties than the first material to form a second layer; and
curing the first material to form a first cured material and curing the second material to form a second cured material,
wherein the first material is arranged to form an outer layer of the multilayer dental restoration and the second material is arranged to form an inner layer of the multilayer dental restoration.

33. The method of claim 32, wherein the inner layer has a hardness that is lower than a hardness for the outer layer.

34. The method of claim 32, wherein the inner layer has an elasticity that is higher than an elasticity of the outer layer.

35. The method of claim 32, wherein the method comprises additively printing a plurality of first layers and a plurality of second layers.

36.-115. (canceled)
